(19) United States
(12) Patent Application Publication
(10) Pub. No.: US 2015/0133558 A1
(43) Pub. Date: May 14, 2015

(54) USE OF N-ACETYLCYSTEINE AMIDE FOR DECREASING INTRACRANIAL PRESSURE

(71) Applicant: Glenn A. Goldstein, New York, NY (US)

(72) Inventor: Glenn A. Goldstein, New York, NY (US)

(21) Appl. No.: 14/514,932

(22) Filed: Oct. 15, 2014

Related U.S. Application Data
(60) Provisional application No. 61/891,037, filed on Oct. 15, 2013.

(51) Int. Cl. A61K 31/16

(52) U.S. Cl. CPC A61K 31/16
USPC 514/616

(57) ABSTRACT

This disclosure describes methods of use for N-acetylcysteine amide for the treatment of penetrating head injury.

### CROSS-REFERENCE TO RELATED APPLICATIONS

This application claims priority to U.S. Provisional Application Ser. No. 61/891,037, filed Oct. 15, 2013, which is incorporated herein by reference in its entirety.

### BACKGROUND

Intracranial hypertension (IH) can be divided into two categories: acute IH and chronic IH. Acute IH often occurs as the result of severe head injury or intracranial bleeding from an aneurysm or a stroke. It is characterized by a very rapid onset after the initial injury and extremely high intracranial pressure that can be fatal. The underlying cause of acute IH is brain-swelling or intracranial bleeding into the sub-arachnoid space that surrounds the brain. In many cases, a piece of skull is surgically removed to accommodate brain swelling and lower intracranial pressure.

In contrast, chronic intracranial hypertension is a neurological disorder in which the increased cerebrospinal fluid (CSF) pressure has generally arisen and remains elevated over a sustained period of time. It can either occur without a detectable cause (idiopathic intracranial hypertension) or be triggered by an identifiable cause such as an underlying disease or disorder, injury, drug or cerebral blood clot (secondary intracranial hypertension).

It is frequently a life-long illness with significant physical, financial and emotional impact. Chronic IH can cause both rapid and progressive changes in vision. Vision loss and blindness due to chronic IH are usually related to optic nerve swelling (papilledema), which is caused by high CSF pressure on the nerve and its blood supply.

It has been postulated that free radicals are linked to increases in intracranial pressure. Various mechanisms have been described to promote free radical production, including glutamate release, intracellular calcium overload, increase in arachidonic acid and its metabolites, hemoglobin denaturation, and iron ion release. However, anti-oxidants, like n-acetylcysteine (NAC) have previously been tried to decrease intra-cranial pressure with no success. (Thomale et al. Intensive Care Med., 32: 149-155 (2006).

There is a need in the art for compounds and therapeutic aspects to treat IH both in its acute and chronic forms. The disclosure below provides such therapeutics.

### SUMMARY

The disclosure provides a method of reducing intracranial pressure in a subject in need thereof comprising administering to the subject a composition comprising a therapeutically effective amount of N-acetylcysteine amide (NACA). In one embodiment, the composition also includes a pharmaceutically acceptable salt or excipient. In another embodiment, the subject suffers from intracranial hypertension. The intracranial hypertension can be acute or chronic intracranial hypertension.

In certain embodiments, the NACA is administered systemically. In other embodiments the NACA is administered intraperitoneally, intravenously, orally or transdermally. In some embodiments, the subject is a mammal. Optionally, the mammal is a human.

In some embodiments, the NACA is administered at between 5 and 10,000 mg/kg, 50 and 500 mg/kg or 200 and 400 mg/kg to the subject. In other embodiments, the subject suffers from stroke, cerebral clots, kidney failure, liver failure, sleep apnea, steroid withdrawal, meningitis, Lyme disease, human immunodeficiency virus infection, acquired immunodeficiency syndrome, poliomyelitis, coxsackie B viral encephalitis, Guillain-Barre syndrome, infectious mononucleosis, syphilis, malaria, lupus, sarcoidosis, hypoparathyroidism, Addison's disease and Bechet disease. In yet other embodiments the subject suffers an overdose of tetracycline, micocycline, isotretinoin, all-trans retinoic acid, Vitamin A, amiodarone, nitofurantoin, lithium, levonorgestral or growth hormone.

### BRIEF DESCRIPTION OF THE FIGURES

FIG. 1 depicts the intracranial pressure over time in rats exposed to three blasts at ˜100 kPa intensity separated by 30 minutes. It was found that such exposure results in both immediate and delayed increases in ICP. Daily measurements on subsequent days indicated a gradual return of ICP to its near pre-blast values. (P: Pre-blast; B1: Blast 1; B2: Blast 2; B3: Blast 3; 1H-3H: 1-3 hours post-blast; D1-D5: Days 1-5 post-blast.)

### DETAILED DESCRIPTION

The present invention provides the use of N-acetylcysteine amide (NAC amide or NACA) or derivatives thereof, or a physiologically acceptable derivative, salt, or ester thereof, to decrease intracranial pressure (ICP). In certain embodiments, methods are provided for treating intracranial hypertension in a subject in need thereof by administering NACA. Optionally, the IH can be acute or chronic IH.

As used herein, a “subject” within the context of the present invention encompasses, without limitation, mammals, e.g., humans, domestic animals and livestock including cats, dogs, cattle and horses. A “subject in need thereof” is a subject having one or more manifestations of disorders, conditions, pathologies, and diseases as disclosed herein in which administration or introduction of NAC amide or its derivatives would be considered beneficial by those of ordinary skill in the art.

“Therapeutic treatment” or “therapeutic effect” means any improvement in the condition of a subject treated by the methods of the present invention, including obtaining a preventative or prophylactic effect, or any alleviation of the severity of signs or symptoms of a disorder, condition, pathology, or disease or its sequelae, including those caused by other treatment methods (e.g., inflammation), which can be detected by means of physical examination, laboratory, or instrumental methods and considered statistically and/or clinically significant by those skilled in the art.

“Prophylactic treatment” or “prophylactic effect” means prevention of any worsening in the condition of a subject treated by the methods of the present invention, as well as prevention of any exacerbation of the severity of signs and symptoms of a disorder, condition, pathology, or disease or its sequelae, including those caused by other treatment methods, which can be detected by means of physical examination, laboratory, or instrumental methods and considered statistically and/or clinically significant by those skilled in the art.

Another aspect of the present invention provides a compound of the formula I:

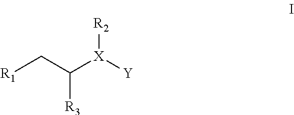

wherein: R₁ is OH, SH, or S—S—Z; X is C or N; Y is NH₂, OH, CH₃—C═O, or NH—CH₃; R₂ is absent, H, or ═O R₃ is absent or

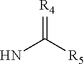

wherein: R₄ is NH or O; R₅ is CF₃, NH₂, or CH₃

and wherein: Z is

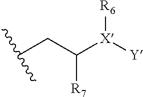

with the proviso that if R₁ is S—S—Z, X and X′ are the same, Y and Y′ are the same, R₂ and R₆ are the same, and R₃ and R₇ are the same.

The present disclosure also provides a NAC amide compound and NAC amide derivatives comprising the compounds disclosed herein. Other derivatives are disclosed in U.S. Pat. No. 8,354,449, incorporated by reference in its entirety.

In another aspect, a process for preparing an L- or D-isomer of the compounds of the present invention are provided, comprising adding a base to L- or D-cystine diamide dihydrochloride to produce a first mixture, and subsequently heating the first mixture under vacuum; adding a methanolic solution to the heated first mixture; acidifying the mixture with alcoholic hydrogen chloride to obtain a first residue; dissolving the first residue in a first solution comprising methanol saturated with ammonia; adding a second solution to the dissolved first residue to produce a second mixture; precipitating and washing the second mixture; filtering and drying the second mixture to obtain a second residue; mixing the second residue with liquid ammonia and an ethanolic solution of ammonium chloride to produce a third mixture; and filtering and drying the third mixture, thereby preparing the L- or D-isomer compound.

In some embodiments, the process further comprises dissolving the L- or D-isomer compound in ether; adding to the dissolved L- or D-isomer compound an ethereal solution of lithium aluminum hydride, ethyl acetate, and water to produce a fourth mixture; and filtering and drying the fourth mixture, thereby preparing the L- or D-isomer compound.

Another aspect of the invention provides a process for preparing an L- or D-isomer of the compounds disclosed herein, comprising mixing S-benzyl-L- or D-cysteine methyl ester hydrochloride or O-benzyl-L- or D-serine methyl ester hydrochloride with a base to produce a first mixture; adding ether to the first mixture; filtering and concentrating the first mixture; repeating steps (c) and (d), to obtain a first residue; adding ethyl acetate and a first solution to the first residue to produce a second mixture; filtering and drying the second mixture to produce a second residue; mixing the second residue with liquid ammonia, sodium metal, and an ethanolic solution of ammonium chloride to produce a third mixture; and filtering and drying the third mixture, thereby preparing the L- or D-isomer compound.

Decrease of Intracranial Pressure and Treatment of Intracranial Hypertension

According to certain embodiments, NACA and derivatives thereof are used to decrease intracranial pressure (ITC). In certain embodiments, NACA and derivatives thereof are used to decrease the ITC of subjects with elevated ITC. ITC is normally 7-15 mm Hg in a supine human. The upper level of ITC is between 20-25 mm Hg. In certain embodiments, a therapeutically effective amount of NACA lowers the ITC to normal levels between 7 and 25 mm Hg. In other embodiments, therapeutically effective amounts of NACA lower ITC, but not to optimum levels. For example, administration of a therapeutically effective amount of NACA to a subject with IH can result in an ITC of between 25 and 500, 50 and 400, 100 and 300 or 150 and 200 mm Hg.

In certain embodiments, subjects suffer from intracranial hypertension (IH) and administration of a therapeutically effective amount of NACA reduces the eleveated ITC associated with IH. In some embodiments, the ITC is reduced to normal levels. In other embodiments, the ITC is reduced to 2-5 times normal levels. In other embodiments, the ITC is reduced to 10-100% greater than normal levels. In other embodiments, the ITC is reduced to 10-30, 30-50, 50-70 or 70-90% of normal levels. Optionally, IH is acute or chronic.

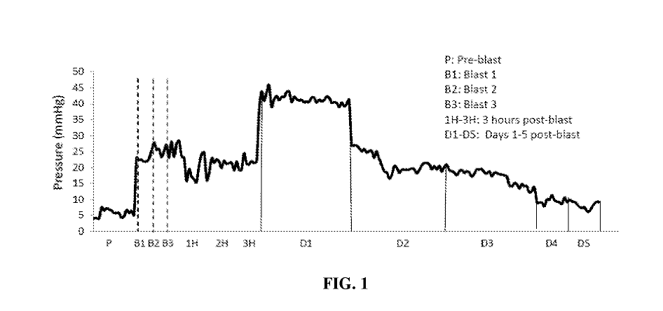

Elevated ITC can have acute causes (acute IH) or be elevated through chronic etiologies (chronic IH). Examples of causes of acute ITC include traumatic brain injury (TBI), aneurysm, stroke or cerebral blood clots. Often, with acute ICP, there is an initial increase in ICP, followed by a later further increase in ICP. An example of this is shown in FIG. 1. In certain embodiments, the first increase in ICP occurs from immediately after injury to 3-24 hours later. In other embodiments, the first increase in ICP occurs from immediately after injury to 5-20, 7-18, 9-16 or 11-14 hours later. In other embodiments, the first increase in ICP occurs from immediately after injury to 1, 2, 3, 4, 5, 6, 7, 8, 9, 10, 11, 12, 13, 14, 15, 16, 17, 18, 19, 20, 21, 22, 23, 24 or more hours later.

In certain embodiments, the second increase in ICP occurs subsequent to the first increase in ICP with no time period between where ICP decreases. In other embodiments, there is a time period of decreased ICP between the first and second ICP increases. This period of decreased ICP can span 3-24 hours. In other embodiments, this period of decreased ICP can span 5-20, 7-18, 9-16 or 11-14 hours. In other embodiments, this period of decreased ICP can span 1, 2, 3, 4, 5, 6, 7, 8, 9, 10, 11, 12, 13, 14, 15, 16, 17, 18, 19, 20, 21, 22, 23 or 24 hours.

In certain embodiments, the second increase in ICP lasts for 1 hour to 5 days before decreasing. In other embodiments, the second increase in ICP lasts 2 hours to 4 days, 10 hours to 3 days, 15 hours to 2 days or 20-24 hours. In other embodiments, the second increase in ICP lasts 5-24 hours.

In some embodiments, the first increase in ICP is a 100-1000% increase from normal ICP. In other embodiments, the first increase in ICP is a 200-900, 300-700 or a 300-500% increase from normal ICP. In other embodiments, the first increase in ICP is about a 100, 200, 300, 400, 500, 600, 700, 800, 900 or 1000% increase from normal ICP.

In some embodiments, the second increase in ICP is a 500-2000% increase from normal ICP. In other embodiments, the second increase in ICP is a 500-1500, 700-1200 or a 800-1100% increase from normal ICP. In other embodiments, the second increase in ICP is about a 500, 600, 700, 800, 900, 1000, 1100, 1200, 1300, 1400, 1500, 1600, 1700, 1800, 1900 or 2000% increase from normal ICP.

In certain embodiments, administration of NACA or derivatives thereof, results in a decrease of the ICP in the first and/or second ICP increase. In certain embodiments, both the first and second ICP increases are prevented by administering NACA prior to the cause of the ICP. In other embodiments, one or both of the first and second increases are reduced. According to certain embodiments, the first and/or second increases in ITC are reduced to 10-100% greater than normal levels. In other embodiments, the first and/or second increases in ITC is reduced to 10-30, 30-50, 50-70 or 70-90% of normal levels.

Doses, amounts or quantities of NACA, or derivatives thereof, are determined on an individual basis. As is appreciated by the skilled practitioner in the art, dosing is dependent on the severity and responsiveness of the OP to be treated, but will normally be one or more doses per day, with course of treatment lasting from one dose to several months, or until a cure is effected or a diminution of disease state is achieved. Persons ordinarily skilled in the art can easily determine optimum dosages, dosing methodologies and repetition rates. For example, a pharmaceutical formulation for orally administrable dosage form can comprise NACA, or a pharmaceutically acceptable salt, ester, or derivative thereof in an amount equivalent to at least 25-500 mg/kg per dose, or in an amount equivalent to at least 50-350 mg/kg per dose, or in an amount equivalent to about 200-300 mg/kg per dose, or about 250 mg/kg per dose. NACA can be administered once or more daily. According to certain embodiments, NACA is administered 1, 2, 3, 4, 5, 6, 7, 8, 9, 10, 11, 12 or more times daily. In certain embodiments, a full dosage is administered every time NACA is administered. In other embodiments, the full dose is split amount multiple administrations over the course of a day.

According to other embodiments, NACA is administered for 1-10 days following a finding of elevated ITC in a subject. In other embodiments, NACA is administered prior to occurrence of elevated ITC. NACA can be administered prophylactically to subjects who are in a situation of high risk of ITC. In certain embodiments, the subjects are at high risk for traumatic brain injury. According to other embodiments, the subjects can be athletes, soldiers, miners, sailors, fishermen, law enforcement, emergency response or firefighters or performing any duty that has an increased risk of elevated ITC. In other embodiments, NACA is administered for 2-8 or 5-7 days in subjects suffering from acute IH either before, during or after occurrence of elevated ITC. For subjects suffering from chronic IH, NACA can be chronically administered for weeks, months or years. NACA dosages can also be increased or decreased depending on the course of ITC in a subject over time. NAC amide or a derivative thereof can be administered to both human and non-human mammals. It therefore has application in both human and veterinary medicine.

Combination Therapies

NACA can be combined with other therapeutics to lower ITC in mammalian subjects. In certain embodiments, NACA is combined with one or more of acetazolamide, methazolamide, furosemide and topiramate. NACA can also be combined with therapeutics for chronic headache such as tricyclic anti-depressants, beta-blockers and calcium channel blockers. NACA can also be combined with corticosteroids or prescription pain medication for the treatment of IH.

Pharmaceutical Compositions

As used herein the term “pharmaceutical composition” refers to a preparation of one or more of the components described herein, or physiologically acceptable salts or prodrugs thereof, with other chemical components such as physiologically suitable carriers and excipients. The purpose of a pharmaceutical composition is to facilitate administration of a compound to an organism. The term “prodrug” refers a precursor compound that can hydrolyze, oxidize, or otherwise react under biological conditions (in vitro or in vivo) to provide the active compound.

The term “excipient” refers to an inert or inactive substance added to a pharmaceutical composition to further facilitate administration of a compound. Non-limiting examples of excipients include calcium carbonate, calcium phosphate, various sugars and types of starch, cellulose derivatives, gelatin, vegetable oils and polyethylene glycols.

The pharmaceutical compositions of the present invention comprise NAC Amide or derviate thereof and may also include one or more additive drugs (e.g., additional active ingredients), such as, but not limited to those listed above that may be suitable for combination therapy.

The pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or by lyophilizing processes.

The compositions for use in accordance with the present invention thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

The term “administration” or any variation thereof as used herein is meant any way of administration. The one or more of NAC Amide or derivatives thereof and at least one additional drug may be administered in one therapeutic dosage form or in two separate therapeutic dosages such as in separate capsules, tablets or injections. In the case of the two separate therapeutic dosages, the administration may be such that the periods between the administrations vary or are determined by the practitioner. It is however preferred that the second drug is administered within the therapeutic response time of the first drug. The one or more of NAC Amide or derivative thereof and at least one additional drug which may be administered either at the same time, or separately, or sequentially, according to the invention, do not represent a mere aggregate of known agents, but a new combination with the valuable property that the effectiveness of the treatment is achieved at a much lower dosage of said at least one additional drug.

The pharmaceutical compositions of the present invention may be administered by any convenient route, for example, by infusion or bolus injection, by absorption through epithelial or mucocutaneous linings (e.g., oral mucosa, rectal and intestinal mucosa, etc.) and may be administered together with any other therapeutic agent. Administration can be systemic or local.

Various delivery systems are known, e.g., encapsulation in liposomes, microparticles, microcapsules or capsules, that may be used to administer the compositions of the invention. Methods of administration include but are not limited to intradermal, intramuscular, intraperitoneal, intravenous, subcutaneous, epidural, oral, sublingual, intranasal, intracerebral, intravaginal, transdermal, rectally (including by suppository or enema), by inhalation, or topically to the cars, nose, eyes, or skin. The preferred mode of administration is left to the discretion of the practitioner, and will depend in part upon the site of the medical condition and the severity of thereof.

For example, for injection the composition of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example DMSO, or polyethylene glycol are generally known in the art.

For oral administration, the composition can be formulated readily by combining the active components with any pharmaceutically acceptable carriers known in the art. Such “carriers” may facilitate the manufacture of such as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active components may be dissolved or suspended in suitable liquids, such as fatty oils, liquid paraffin, or liquid polyethylene glycols.

Dyestuffs or pigments may be added to the tablets or dragee coatings for identification or to characterize different combinations of active NSAID doses. In addition, stabilizers may be added.

Pharmaceutical compositions for parenteral administration include aqueous solutions of the active preparation in a water-soluble form. Additionally, suspensions of the active preparation may be prepared as oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl, cellulose, sorbitol or dextran. Optionally, the suspension may also contain suitable stabilizers or agents, which increase the solubility of the compounds, to allow for the preparation of highly concentrated solutions.

Alternatively, the composition may be in a powder form for constitution before use with a suitable vehicle, e.g., sterile, pyrogen-free water. The exact formulation, route of administration and dosage may be chosen by the physician familiar with the patient's condition. (See for example Fingl, et al., 1975, in “The Pharmacological Basis of Therapeutics”, Chapter I, p. 1). Depending on the severity and responsiveness of the condition treated, dosing can also be a single administration of a slow release composition, with course of treatment lasting from several days to several weeks or until cure is effected or diminution of the disease state is achieved.

### EXAMPLES

### Example 1

### NACA Reduces Intracranial Pressure in Rats

It is well accepted that oxidative damage plays a key role in open and closed head injuries and oxidative stress has been also implicated in pathophysiological mechanisms of blood brain barrier (BBB) damage. The first goal of this study is to determine the time-dependent progression of BBB damage, brain edema, intracranial pressure (ICP) variations and correlation among them in blast injured rats. In our preliminary studies, in which rats were exposed to three blasts of ˜100 kPa intensity separated by 30 minutes, it was found that such exposure results in both immediate and delayed increases in ICP (FIG. 1). Daily measurements on subsequent days indicated a gradual return of ICP to its near pre-blast values. It appears that measurement of ICP following varying intensities of blast enables characterization of blast brain injury by defining the blast thresholds for damaging effects. It could also be an indicator of the time for optimal pharmacological interventions for treatment.

In the second step, we use the antioxidant NACA to test the hypothesis that the increase in ICP and BBB impairment after blast is caused by accumulation of reactive oxygen radical (ROS) and ROS-mediated damage to BBB Animal studies have demonstrated that NACA is more efficient in crossing the blood brain barrier and restoring intracellular thiols than N-acetylcysteine. We previously demonstrated that NACA has potential to benefit other symptoms associated with blast injury of “hollow” organs such as the lungs.

This study will be divided into three main tasks: 1) assessment of changes in ICP following exposure to blast; 2) histological evaluation of BBB permeability, ultrastructural changes in choroid plexus, and brain edema; and 3) determining the effect of antioxidant NACA on ICP increase and BBB permeability. Male Sprague-Dawley rats weighing 250-300 g will be utilized in this study. All animals will be treated in accordance with the Animal Care and Use Committee approved protocols. The blast brain injury will be induced by exposing anesthetized rats to BOP generated and transmitted in a shock tube.

Specific Aim #1: Characterization of ICP Increase Following Exposure to Blast

The aim of this study will be to determine the time course of ICP increase after exposure to blast and to assess the threshold for blast intensity to produce the damaging effect. Two intensities of blast in a single or multiple exposures at ˜60 or 100 kPa will be used to correlate the response of ICP to blast.

ICP measurements: Long-term ICP measurements are carried out by implanting a telemetric device in the rats. The device casing, approximately 10 mm in diameter, contains the electronics necessary for signal conditioning and wireless transmission. A MEMS-based pressure sensor will be placed at the base of the casing at the site of a conduit, (approximately 1 mm in diameter and 3-4 mm long) emanating from the base.

Implantation procedure: The implantation procedure is conducted in sterile conditions under isoflurane anesthesia. A 1 mm hole is drilled in the parietal region (0.9 mm lateral and 1.5 mm posterior to bregma). The conduit of the ICP device is tunneled through the hole for its placement in the intra-ventricular space. The casing is cemented to the skull with bone cement to ensure a secure implantation.

Exposures and data recording: Wireless data is recorded continuously by a receiver chip antenna connected to a digital phosphor oscilloscope (DPO 7254, Tektronix). ICP is recorded for 10-15 minutes prior to blast to determine baseline ICP for the test animal. Animals are exposed to single or multiple blasts (separated by 1 day) to induce brain injury. ICP is recorded immediately after blast for 3-4 hours and on a daily basis for 10 days (Table 1). Total number of animals in Specific Aim #1=96.

Data analysis: Data analysis is carried out on a MATLAB platform and will include signal processing such as filtering, frequency analysis and calibration for determination of ICP.

TABLE 1
Grouping of animals for ICP study.
ICP Study

| Blast Schedule | | |
|---|---|---|
| Day 1 | Day 2 | Day 3 |

Blast Intensity (Mild): 60-70 kPa Time of sacrifice: 10 days after last blast

| | | | | |
|---|---|---|---|---|
| Group A1 (One blast) | X | | | 8 blasted and 8 |
| Group A2 (Two blasts) | X | X | | control animals in |
| Group A3 (Three blasts) | X | X | X | each group; |
| | | | | N = 48 |

Blast intensity (Moderate): 100-120 kPa Time of sacrifice: 10 days
after last blast

| | | | | |
|---|---|---|---|---|
| Group B1 (One blast) | X | | | 8 blasted and 8 |
| Group B2 (Two blasts) | X | X | | control animals in |
| Group B3 (Three blasts) | X | X | X | each group; |
| | | | | N = 48 |

Specific Aim #2: Kinetics of BBB Damage

The aim of these experiments is to evaluate the time-dependent change in the integrity of the BBB after brain injury and its correlation with ICP changes. BBB integrity is quantified by: 1) assessing the intensity of fluorescently labeled dextrans of varying molecular weights that leak into the brain parenchyma, and 2) identifying histopathologic alterations in choroid plexus structure. Kinetics of BBB opening (immediate and delayed after blast) are examined at one intensity level of blast (100 kPa) and correlated with the increase of ICP.

Injection of Fluorescently labeled Dextran: Animals are injected with Fluorescein isothiocyanate (FITC)—Dextran 4 kDa (Sigma) (10 mg/rat) in 300 μl of 0.9% NaCl and tretramethylrodamine isothiocyanate (TRITC)-Dextran 40 kDa (Sigma) at (10 mg/rat) in 300 μl of 0.9% NaCl by tail vein injection. One group of animals receives dextran injection 30 min before blast and sacrificed 1 hour after exposure. Other groups of animals receive dextran injection and will be sacrificed 1, 2, 3 or 7 days after blast.

Tissue fixation and sampling: At the determined intervals commencing one hour after dextran injection animals are euthanized with an overdose of pentobarbital and intracardially perfused with PBS followed by 4% (wt/vol) paraformaldehyde in PBS. Brains are quickly removed from the skull and post fixed in 4% (wt/vol) paraformaldehyde in PBS overnight.

Immunohistochemistry: 30 μm-thick sections of the entire forebrain in the coronal plane are prepared using a freezing sliding microtome. The preserved sections are used for immunocytochemistry for light microscopy or immunofluorescence staining for fluorescent microscopy. Staining of sections utilizes a free floating method with the section mounted on glass slides and cover-slipped with the appropriate fixative. A high powered bright field microscope and a high powered fluorescent scope like the Nikon Eclipse E800 is utilized for analysis of the specimen slides.

Grouping of animals for BBB and Brain Edema Study: Two (2) blast groups with 1 or 3 blast exposures at 100 kPa separated by one day and five (5) sampling intervals: 1 h and 1, 2, 3 and 7 days after last blast. Total animals in Specific Aim #2: 5 animals in each group×2×5=50 animals.

Specific Aim #3: Involvement of Oxidative Stress in BBB Dysfunction and ICP Increase after Exposure to Blast.

The group with the highest response in ICP and BBB changes, most likely exposed to 100 kPa, is injected with a bolus i.p. injection of the sample antioxidant drug NACA 30 min after exposure or at the time with the secondary ICP increase. A single dose of NACA (250 mg/kg) is used based on previous results in mice demonstrating inhibition of oxidative damage in brain. The efficacy of NACA is assessed by ICP recordings and BBB permeability detection as described in Specific Aims #1 and #2. Twenty four animals are used for the drug study (12 animals in each of two groups)

### Example 2

### NACA Keeps ICP at Normal Levels in Rats Exposed to Blast

Spinal cord and brain damage from blast was assessed by continuous measuring of intracranial pressure (ICP) and tight junction protein destruction. Previous research has determined that after blast exposure there is a biphasic elevation of ICP, with the initial elevation on day 1, which then decreases to near normal. However on days 3-4 there is a second increase in ICP which is very damaging, and is often lethal. This has been accompanied by destruction of tight junction proteins.

Two groups of 10 rats are exposed to pressure wave. In the control group after several hours there is an initial elevation of ICP which declined and in approximately 3 days there was a second increase in ICP.

The second group was treated with NACA before the blast. Treatment continued for approximately 7 days. In this group ICP remained normal, without elevation for the entire treatment period.

### REFERENCES

1. Warden D. (2006). J. Head Trauma Rehab. 21:398-402.
2. French L M. (2009). J. Head Trauma Rehab. 24:1-3.
3. Ling G S, Ecklund J M. (2011). Curr. Opin. Anaesthesiol. 24:124-130.
4. Beaumont A, Marmarou A, Hayasaki K, Barzo P, Fatauros P, Corwin F, Marmarou C, Dunbar J. (2000). Acta Neurochir. Suppl. 76:125-129.
5. Donkin J J, Vink R. (2010). Curr. Opin Neurol. 23:293-299.
6. Saljo A, Bolouri H, Mayorga M, Svenson B, Hamberger A. (2010). J. Neurotrauma 27:383-9.
7. Hall E D, Detloff M R, Johnson K, Kupina N C. (2004). J. Neurotrauma 21:9-20.
8. Readnower R D, Chavko M, Adeeb S, Conroy M D, Pauly J R, McCarron R M, Sullivan P G. (2010). J. Neurosci. Res. 88:3530-9.
9. Gilgun-Sherki Y, Melamed E, Offen D. (2004). J. Neurol. 251:261-8.
10. Chavko M, Adeeb S, Ahlers S T, McCarron R M. (2009). Shock 32:325-31.
11. Banerjee A, Zhang X, Manda K R, Banks W A, Ercal N. (2010). Free Radical Biol. Med. 48:1388-1398.

What is claimed is:

1. A method of reducing intracranial pressure in a subject in need thereof comprising administering to the subject a composition comprising a therapeutically effective amount of N-acetylcysteine amide (NACA).

2. The method of claim 1, wherein the composition further comprises a pharmaceutically acceptable salt or excipient.

3. The method of claim 1, wherein the subject suffers from intracranial hypertension.

4. The method of claim 3, wherein the intracranial hypertension is acute intracranial hypertension.

5. The method of claim 3, wherein the intracranial hypertension is chronic intracranial hypertension.

6. The method of claim 1, wherein the NACA is administered systemically.

7. The method of claim 5, wherein the NACA is administered intraperitoneally, intravenously, orally or transdermally.

8. The method of claim 1, wherein the subject is a mammal.

9. The method of claim 8, wherein the mammal is a human.

10. The method of claim 1, wherein the NACA is administered at between 5 and 10,000 mg/kg.

11. The method of claim 1, wherein the NACA is administered at between 50 and 500 mg/kg to the subject.

12. The method of claim 11, wherein the NACA is administered at between 200 and 400 mg/kg to the subject.

13. The method of claim 1, wherein the subject suffers from stroke, cerebral clots, kidney failure, liver failure, sleep apnea, steroid withdrawal, meningitis, Lyme disease, human immunodeficiency virus infection, acquired immunodeficiency syndrome, poliomyelitis, coxsackie B viral encephalitis, Guillain-Barre syndrome, infectious mononucleosis, syphilis, malaria, lupus, sarcoidosis, hypoparathyroidism, Addison's disease and Bechet disease.

14. The method of claim 1, wherein the subject suffers an overdose of tetracycline, micocycline, isotretinoin, all-trans retinoic acid, Vitamin A, amiodarone, nitofurantoin, lithium, levonorgestral or growth hormone.
